(19) United States
(12) Patent Application Publication
(10) Pub. No.: US 2023/0141272 A1
(43) Pub. Date: May 11, 2023

(54) DYNAMIC AIR FILTRATION

(71) Applicant: Carrier Corporation, Palm Beach Gardens, FL (US)

(72) Inventors: Siva Krishna Kattinti, Hyderabad (IN); Madhukar Sinha, Narsingpur (IN); Bhogeshwari Irukulla, Hyderabad (IN)

(21) Appl. No.: 17/982,018

(22) Filed: Nov. 7, 2022

Related U.S. Application Data
(60) Provisional application No. 63/276,908, filed on Nov. 8, 2021.

(51) Int. Cl. B01D 46/46; F24F 3/16; F24F 13/28; F24F 11/39; B01D 46/62; B01D 46/90; B01D 53/86; B01D 46/00; A61L 9/18

(52) U.S. Cl. CPC B01D 46/46; F24F 3/16; F24F 13/28; F24F 11/39; B01D 46/62; B01D 46/90; B01D 53/8668; B01D 46/0028; A61L 9/18; F24F 2110/50

(57) ABSTRACT

A filter section of a heating, ventilation, and air conditioning (HVAC) system includes a plurality of filters arranged in series relative to a flow path of air through the filter section. The plurality of filters includes at least one movable filter transformable between an extended position arranged within the flow path of air and a retracted position removed from the flow path of air. At least one movement mechanism is operably coupled to the at least one movable filter to transform the at least one movable filter between the extended position and the retracted position.

### CROSS-REFERENCE TO RELATED APPLICATIONS

This application claims the benefit of U.S. Provisional Application No. 63/276,908 filed Nov. 8, 2021, the disclosure of which is incorporated herein by reference in its entirety.

### BACKGROUND

Embodiments of the present disclosure relate generally to heating, ventilation, and air conditioning (HVAC) systems, and more particularly to an indoor air quality system for use in an HVAC system.

Heating, ventilation, and air conditioning (HVAC) systems are used to deliver a flow of conditioned air to one or more areas within a building. In addition to conditioning the temperature and/or humidity of the air, HVAC systems typically includes a filtration system configured to maintain an acceptable level of air quality within the areas being conditioned. The filtration system includes commonly includes a series of filters which removes airborne contaminants, such as smoke, volatile organic compounds, and debris from the air being circulated therethrough. While existing filtration systems are effective at removing undesired pollutants from an airflow, such filtration systems can also cause a pressure drop within the HVAC system, particularly as particles being to accumulate at the filters, thereby reducing the flow through the filtration system. To compensate for the resulting drop in pressure, the fan consumes additional energy.

### BRIEF DESCRIPTION

According to an embodiment, a filter section of a heating, ventilation, and air conditioning (HVAC) system includes a plurality of filters arranged in series relative to a flow path of air through the filter section. The plurality of filters includes at least one movable filter transformable between an extended position arranged within the flow path of air and a retracted position removed from the flow path of air. At least one movement mechanism is operably coupled to the at least one movable filter to transform the at least one movable filter between the extended position and the retracted position.

In addition to one or more of the features described herein, or as an alternative, further embodiments the plurality of filters further comprises a filter that is fixedly mounted within the flow path of air.

In addition to one or more of the features described herein, or as an alternative, further embodiments the filter that is fixedly mounted is arranged upstream from the at least one movable filter.

In addition to one or more of the features described herein, or as an alternative, further embodiments the filter that is fixedly mounted is a pre-filter.

In addition to one or more of the features described herein, or as an alternative, further embodiments the filter that is fixedly mounted is arranged downstream from the at least one movable filter.

In addition to one or more of the features described herein, or as an alternative, further embodiments the filter that is fixedly mounted is an active filter.

In addition to one or more of the features described herein, or as an alternative, further embodiments the at least one movable filter is translatable between the extended position and the retracted position.

In addition to one or more of the features described herein, or as an alternative, further embodiments the at least one movable filter is rotatable between the extended position and the retracted position.

In addition to one or more of the features described herein, or as an alternative, further embodiments the at least one movable filter further comprises a plurality of movable filters, wherein a configuration of the plurality of movable filters varies.

In addition to one or more of the features described herein, or as an alternative, further embodiments the at least one movement mechanism includes a plurality of movement mechanisms, each movement mechanism being operably coupled to one of the plurality of movable filters.

In addition to one or more of the features described herein, or as an alternative, further embodiments the at least one movement mechanism is operably coupled to the plurality of movable filters.

According to an embodiment, a heating, ventilation, and air conditioning (HVAC) system includes a filter section configured to receive air. The filter section includes at least one movable filter transformable between an extended position arranged within a flow path of the air and a retracted position removed from the flow path of the air. The HVAC system additionally includes at least one sensor for monitoring an air quality of the air and a controller operably coupled to the at least one sensor and to the at least one movable filter. The controller is configured to move the at least one movable filter between the extended position and the retracted position in response to the air quality of the air.

In addition to one or more of the features described herein, or as an alternative, further embodiments the at least one sensor is arranged upstream from the filter section.

In addition to one or more of the features described herein, or as an alternative, further embodiments the at least one movable filter further comprises a plurality of movable filters and a configuration of the plurality of movable filters varies.

In addition to one or more of the features described herein, or as an alternative, further embodiments the controller is configured to select at least one of the plurality of movable filters to arrange within the flow path in response to the air quality measured by the at least one sensor.

According to an embodiment, a method of adjusting a configuration of a filter section of a heating, ventilation, and air conditioning (HVAC) system includes sensing an air quality of a flow of air upstream from the filter section, identifying at least one of a plurality of movable filters to apply to the flow of air to achieve preset air quality limits of the HVAC system, and moving the at least one movable filter identified into the flow of air.

In addition to one or more of the features described herein, or as an alternative, further embodiments include moving another of the plurality of movable filters out of the flow of air.

In addition to one or more of the features described herein, or as an alternative, further embodiments include identifying the at least one of the plurality of movable filters further comprises comparing the air quality of the flow of air with the preset air quality limits.

In addition to one or more of the features described herein, or as an alternative, further embodiments include sensing the air quality of the flow of air downstream from the filter section, sensing the air quality of an outdoor air provided to the HVAC system, and adjusting a position of an outside air damper in response to sensing the air quality of the outdoor air.

In addition to one or more of the features described herein, or as an alternative, further embodiments include turning on an active filter.

### BRIEF DESCRIPTION OF THE DRAWINGS

The following descriptions should not be considered limiting in any way. With reference to the accompanying drawings, like elements are numbered alike:

FIG. 1 is a schematic diagram of a heating, ventilation, and air conditioning (HVAC) system according to an embodiment;

FIG. 2A is a schematic cross-section view of a movable filter in an extended position according to an embodiment;

FIG. 2B is a schematic cross-section view of a movable filter in an retracted position according to an embodiment; and

FIG. 3 is a flow diagram of a method of adjusting the configuration of a filter section of the HVAC system according to an embodiment.

### DETAILED DESCRIPTION

A detailed description of one or more embodiments of the disclosed apparatus and method are presented herein by way of exemplification and not limitation with reference to the Figures.

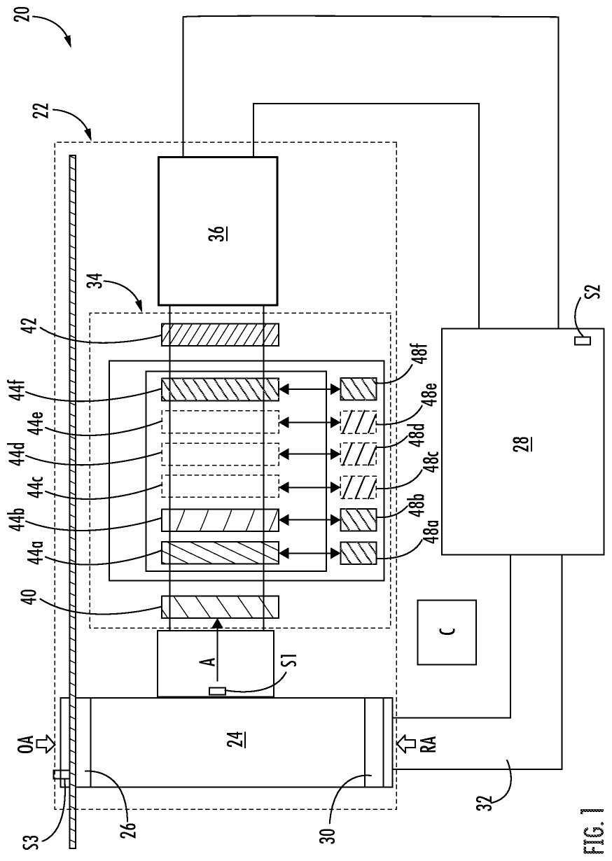

With reference now to FIG. 1, a schematic diagram of a ducted heating ventilation and air conditioning (HVAC) system 20, such as a dedicated outdoor air system for example, is illustrated. In an embodiment, the HVAC system 20 shown includes an air handling unit, illustrated schematically at 22, such as roof top unit for example. The air handling unit 22 includes a mixing duct 24 configured to receive a supply of outside air OA via operation of an outside air damper 26. In some embodiments, the mixing duct 24 may alternatively or additionally be configured to receive a supply or return air RA from one or more of the zones 28 of the HVAC system 20, such as via return air damper 30 coupled to a return air duct 32 extending between the at least one zone 28 and the air handling unit 22 for example. Although only a single zone 28 or area to be conditioned by the HVAC system 20 is illustrated in the FIG., it should be understood that an HVAC system 20 having any number of zones 28, such as at least two zones, at least three zones, or at least five zones for example, is within the scope of the disclosure.

From the mixing duct 24, the outside air OA or the mixture of outside air and return air (OA+RA), referred to herein generally as “air” A, is configured to pass through a filter section, illustrated at 34, having one or more filters arranged therein. Arranged downstream from the filter section 34 relative to the flow of the air A are a plurality of components, represented schematically at 36, configured to condition the air before the air is supplied to the one or more zones 28. These air conditioning components may include a coil unit and a fan, such as a variable speed fan for example, configured to supply air to the coil unit. Return air RA is drawn from the one or more zones 28 into the return air duct 32. The return air within the return duct 32 may be provided to the mixing duct 24, or alternatively or additionally, may be exhausted to the atmosphere surrounding the air handling unit 22. It should be understood that the HVAC system 20 illustrated and described herein is intended as an example only and that any suitable HVAC system 20 is within the scope of the disclosure.

With continued reference to FIG. 1, the filter section 34 includes a plurality of filters arranged in series relative to the flow path of the air A therethrough. In an embodiment, the plurality of filters includes a pre-filter 40, such as arranged at the upstream end of the filter section 34. The pre-filter 40 may be configured to remove large unwanted particles from the air, such as dust, hair, insects, pollen, and fibers. Alternatively, or in addition, the plurality of filters may include an active filter 42. The active filter 42 may be arranged at the downstream end of the filter section 34, such as the last filter within the series of filters. The active filter 42 may be configured to remove volatile organic compounds (VOCs), bacteria, and viruses by producing ions that inactivate or destroy harmful contaminants. For example, the filter may be a photocatalytic filter. The active filter 42 may require power, such as to energize a light source for example, to initiate operation of the filter or to sterilize the filter.

In an embodiment, the plurality of filters includes at least central filter 44 distinct from the pre-filter 40 and the active filter 42. In embodiments of the HVAC system 20 including a pre-filter 40, the at least one central filter 44 is arranged downstream from the pre-filter 40 relative to the flow path of the air A. In embodiments of the HVAC system 20 including an active filter 42, the at least one central filter 44 is located upstream from the active filter 42. Although the illustrated non-limiting embodiment has six central filters 44a-44f, it should be understood that a filter section 34 having any number of central filters is contemplated herein. Further, in embodiments where the filter section 34 includes a plurality of central filters 44, a configuration of the plurality of central filters 44 varies. In an embodiment, each of the plurality of central filters 44 may have a different configurations. For example, one or more parameters of the plurality of central filters 44, such as the size, efficiency, and associated particle size, may vary between the central filters 44.

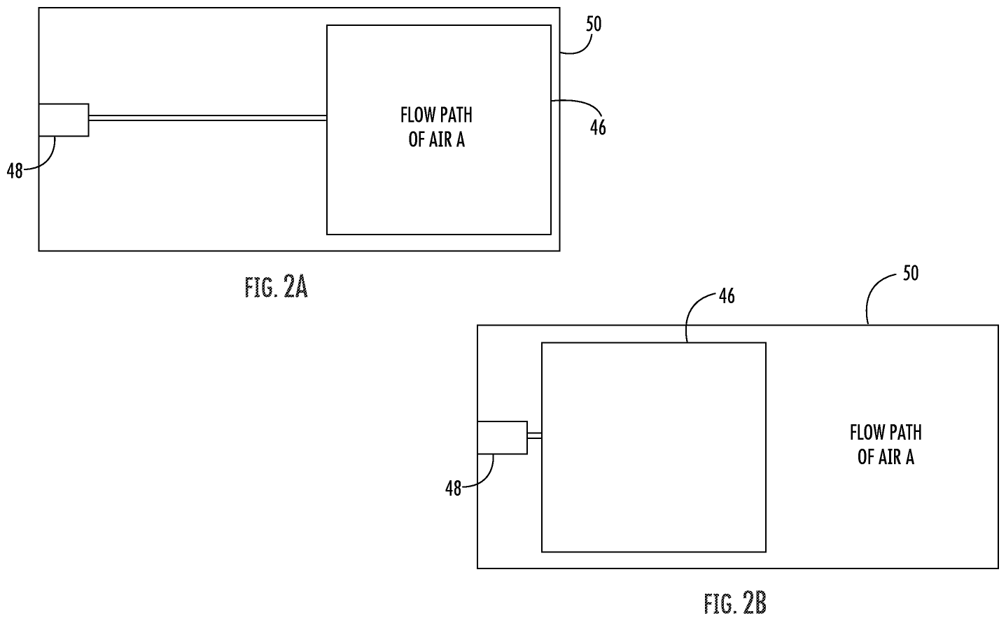

With continued reference to FIG. 1, and further reference to FIGS. 2A and 2B, one or more of the plurality of filters within the filter section 34 is selectively movable between an extended position (FIG. 2A) and a retracted position (FIG. 2B). In the extended position, the at least one movable filter, represented by numeral 46, is arranged within the flow path of the air A and in the retracted position, the at least one movable filter 46 is located outside of or removed from the flow path of air A. In an embodiment, at least one of the central filters 44a-44f is selectively movable in and out of the flow path of air A. Although in the illustrated non-limiting embodiment each of the central filters 44a-44f is selectively movable relative to the flow path of the air A, it should be understood that embodiments where only a portion of the central filters is configured to move is also within the scope of the disclosure. Further, it should be understood that although the pre-filter 40 and the active filter 42 are illustrated as being fixedly mounted within the flow path of air A, in an embodiment, at least one of the pre-filter 40 and the active filter 42 may be selectively movable, alternatively to or in addition to the at least one central filter.

A movement mechanism 48 is operably coupled to the at least one movable filter 46. In embodiments where a plurality of filters are movable relative to the flow path of the air A, a plurality of movement mechanisms (see 48f-48f in FIG. 1) may be coupled to the plurality of movable filters 46, respectively. However, embodiments where a single movement mechanism 48 is operable to move a plurality of filters 46 are also within the scope of the disclosure. The one or more movement mechanisms 48 may be arranged within the interior of the duct 50 containing the at least one movable filter 46 as shown, or alternatively, may be located at an exterior of the duct 50.

In an embodiment, the at least one movement mechanism 48 associated with the at least one movable filter 46 is an actuator, such as a linear actuator for example. Accordingly, the movable filter 46 is configured to translate linearly between the extended and retracted positions. In embodiments where the filter is configured to translate horizontally, as shown in FIGS. 2A and 2B, the width of the portion of the duct 50 containing the one or more movable filters 46 must be at least double the width of the widest movable filter 46 to accommodate the movable filter 46 in both the extended and retracted positions. However, in other embodiments, one or more of the movable filters 46 may be configured to translate vertically. Further, embodiments where one or more movement mechanism 48 is configured to move a filter 46 in another manner, for example, rotate the filter 46 between the extended position and the retracted position, are also within the scope of the disclosure.

The at least one movable filter 46 is transformable between the extended position and the retracted position in response to a detected air quality parameter of the air A to be provided to the filter section 34. In an embodiment, the HVAC system 20 includes a controller C and at least one sensor S operably coupled to the controller C, the at least one sensor S being operable to monitor one or more air quality parameters of the air A. One such sensor, illustrated at S1, may be arranged upstream from the filter section 34 relative to the flow path of the air A, such as at an outlet of the mixing duct 24 for example. In response to the pollutants detected within the air A by the sensor S1, the controller C will determine which of the movable filters 46 should be arranged within the flow path to achieve an air quality that meets the set requirements of the HVAC system 20. The controller C will then send a command to one or more of the movement mechanisms 48 to move one or more of the filters 46 into the flow path. The commands generated by the controller C will not only include commands for movement of one or more movable filters 46 required to be applied to the air into the flow path, but also may include commands for movement of one or more movable filters 46 that no longer need to be used into a retracted position. Accordingly, each of the movable filters 46 is only arranged within the flow path of the air A when needed.

In an embodiment, the HVAC system 20 includes one or more differential air pressure sensors (not shown) associated with the filter section 34 and operably coupled to the controller C. The controller C may be configured to rely on the input from the differential air pressure sensor to determine a pressure drop of the air A within the filter section. In an embodiment, if the pressure drop exceeds a predefined level, the controller C may generate an alarm or other indication that one or more of the filters within the filter section 34 requires cleaning or replacement.

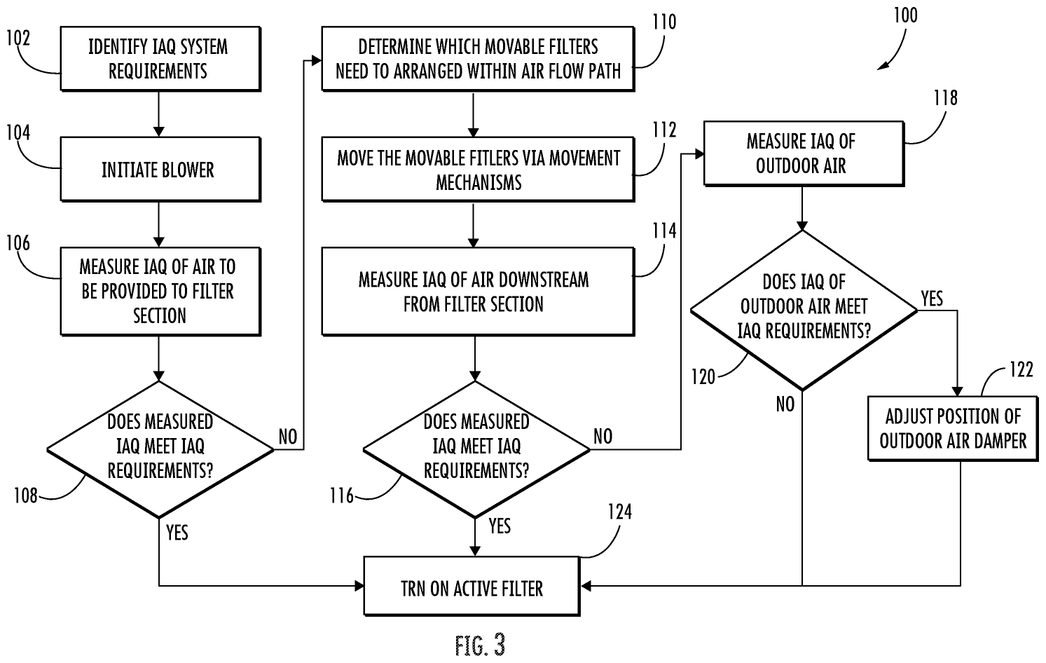

With reference now to FIG. 3, a method of managing the air quality within an HVAC system 20 having one or more movable filters 46 is illustrated. Upon startup of the HVAC system 20, the controller C will identify the preset air quality limits or requirements in block 102, and will then initiate operation of the blower, as shown in block 104, to move the air through the system 20. As the air flows through the HVAC system 20, the sensor Si will measure at least one parameter associated with the air quality (block 106) of the flow of air A. In block 108, the controller C will compare the sensed parameter(s) with the air quality requirements of the HVAC system 20. If the sensed air quality of the air A satisfies the preset requirements, none of the movable filters 46 need to be moved relative to the flow path of the air A. However, if one or more parameters is outside of or exceeds the preset requirements, in block 110, the controller C determines which movable filters 46 are needed to achieve a suitable air quality. The respective filters 46 are then moved into a position within the air flow path, as shown in block 112. This movement may further include removing any movable filter 46 from the flow path that is not necessary to achieve an air quality within the preset limits. Another air quality sensor S2 arranged downstream from the filter section 34, such as arranged within an area to be conditioned 28, is configured to measure at least one parameter associated with air quality in block 114 and communicate these measurements to the controller C. The controller C then compares the sensed air quality of the air A downstream from the filter section 34 with the preset requirements (block 116). If the sensed air quality parameters satisfy the preset requirements, the correct filters are arranged within the flow path.

In embodiments where the sensed air quality parameters are outside of the preset limits, as shown in block 118, one or more air quality parameters of the outdoor air OA provided to the mixing duct 24 via the outside air damper 26 are monitored by another air quality sensor S3. If a controller C determines that the air quality of the outdoor air OA is within the predetermined air quality limits (block 120), a position of the outside air damper 26 will be adjusted to increase the fresh or outdoor air OA provided to the mixing duct 24 (see block 122). However, in the event that the sensed air quality of the outdoor air OA does not meet the preset requirements, the outside air damper 26 will not be adjusted. Accordingly, after each of determining that sensed air quality of the air A upstream from filter section 34 is satisfactory, determining that the sensed air quality of the air A downstream from the filter section 34 is satisfactory, determining that the air quality of the outdoor air OA is not satisfactory, and adjusting the position of the outside air damper 26, in block 124, operation of the active filter 42 is initiated, such as by supplying power to a light source used in combination with the active filter for example.

A filter section 34 of an HVAC system 20 having one or more filters that are movable in and out of the flow path of the air flowing A therethrough will extend the lifetime of the movable filters 46 located within the filter section 34 by limiting their interaction with the air A. Further, by arranging only the filters necessary to achieve a predetermine air quality within the flow path, the overall power consumption of the HVAC system 20 will be reduced.

The term “about” is intended to include the degree of error associated with measurement of the particular quantity based upon the equipment available at the time of filing the application.

The terminology used herein is for the purpose of describing particular embodiments only and is not intended to be limiting of the present disclosure. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, element components, and/or groups thereof.

While the present disclosure has been described with reference to an exemplary embodiment or embodiments, it will be understood by those skilled in the art that various changes may be made and equivalents may be substituted for elements thereof without departing from the scope of the present disclosure. In addition, many modifications may be made to adapt a particular situation or material to the teachings of the present disclosure without departing from the essential scope thereof. Therefore, it is intended that the present disclosure not be limited to the particular embodiment disclosed as the best mode contemplated for carrying out this present disclosure, but that the present disclosure will include all embodiments falling within the scope of the claims.

What is claimed is:

1. A filter section of a heating, ventilation, and air conditioning (HVAC) system comprising:
a plurality of filters arranged in series relative to a flow path of air through the filter section, the plurality of filters including at least one movable filter transformable between an extended position arranged within the flow path of air and a retracted position removed from the flow path of air; and
at least one movement mechanism operably coupled to the at least one movable filter to transform the at least one movable filter between the extended position and the retracted position.

2. The filter section of claim 1, wherein the plurality of filters further comprises a filter that is fixedly mounted within the flow path of air.

3. The filter section of claim 2, wherein the filter that is fixedly mounted is arranged upstream from the at least one movable filter.

4. The filter section of claim 2, wherein the filter that is fixedly mounted is a pre-filter.

5. The filter section of claim 2, wherein the filter that is fixedly mounted is arranged downstream from the at least one movable filter.

6. The filter section of claim 2, wherein the filter that is fixedly mounted is an active filter.

7. The filter section of claim 1, wherein the at least one movable filter is translatable between the extended position and the retracted position.

8. The filter section of claim 1, wherein the at least one movable filter is rotatable between the extended position and the retracted position.

9. The filter section of claim 1, wherein the at least one movable filter further comprises a plurality of movable filters, wherein a configuration of the plurality of movable filters varies.

10. The filter section of claim 9, wherein the at least one movement mechanism includes a plurality of movement mechanisms, each movement mechanism being operably coupled to one of the plurality of movable filters.

11. The filter section of claim 9, wherein the at least one movement mechanism is operably coupled to the plurality of movable filters.

12. A heating, ventilation, and air conditioning (HVAC) system comprising:
a filter section configured to receive air, the filter section including at least one movable filter transformable between an extended position arranged within a flow path of the air and a retracted position removed from the flow path of the air;
at least one sensor for monitoring an air quality of the air; and
a controller operably coupled to the at least one sensor and to the at least one movable filter, wherein the controller is configured to move the at least one movable filter between the extended position and the retracted position in response to the air quality of the air.

13. The HVAC system of claim 12, wherein the at least one sensor is arranged upstream from the filter section.

14. The HVAC system of claim 12, wherein the at least one movable filter further comprises a plurality of movable filters and a configuration of the plurality of movable filters varies.

15. The HVAC system of claim 12, wherein the controller is configured to select at least one of the plurality of movable filters to arrange within the flow path in response to the air quality measured by the at least one sensor.

16. A method of adjusting a configuration of a filter section of a heating, ventilation, and air conditioning (HVAC) system comprising:
sensing an air quality of a flow of air upstream from the filter section;
identifying at least one of a plurality of movable filters to apply to the flow of air to achieve preset air quality limits of the HVAC system; and
moving the at least one movable filter identified into the flow of air.

17. The method of claim 16, further comprising moving another of the plurality of movable filters out of the flow of air.

18. The method of claim 16, wherein identifying the at least one of the plurality of movable filters further comprises comparing the air quality of the flow of air with the preset air quality limits.

19. The method of claim 16, further comprising:
sensing the air quality of the flow of air downstream from the filter section;
sensing the air quality of an outdoor air provided to the HVAC system; and
adjusting a position of an outside air damper in response to sensing the air quality of the outdoor air.

20. The method of claim 16, further comprising turning on an active filter.
